(19) United States
(12) Patent Application Publication
(10) Pub. No.: US 2026/0356925 A1
(43) Pub. Date: Nov. 12, 2026

(54) IMPROVED ECULIZUMAB PRECISION DOSING

(71) Applicant: CHILDREN'S HOSPITAL MEDICAL CENTER, Cincinnati, OH (US)

(72) Inventors: Sonata Jodele, Hawthorne, CA (US); Kana Mizuno, Mason, OH (US); Alexander A. Vinks, Missoula, MO (US)

(21) Appl. No.: 19/135,238

(22) PCT Filed: Dec. 21, 2023
(86) PCT No.: PCT/US2023/085290

§ 371 Date: Jun. 3, 2025

Related U.S. Application Data
(60) Provisional application No. 63/434,184, filed on Dec. 21, 2022.
(60) Provisional application No. 63/434,187, filed on Dec. 21, 2022.

(51) Int. Cl. C07K 16/18; A61K 39/00; A61P 7/02

(52) U.S. Cl. CPC C07K 16/18; A61P 7/02; A61K 2039/505; A61K 2039/54; A61K 2039/545; C07K 2317/24

(57) ABSTRACT

Disclosed herein are methods of treating a human patient having TA-TMA, for example high-risk TA-TMA with MODS, comprising administration of a C5 inhibitor, for example eculizumab or an antigen binding fragment thereof. The disclosed dosing regimens may comprise one or more of a loading phase, induction phase, and maintenance phase. The disclosed methods are particularly advantageous for individuals that do not have clinically relevant bleeding.

### BACKGROUND

Transplant-associated thrombotic microangiopathy (TA-TMA) is a fatal post-transplant complication of hematopoietic stem cell transplantation (HSCT). Untreated patients with high-risk TA-TMA have very high mortality (16.7% 1 year post HSCT and 9% overall survival). Survival for TA-TMA has been improved by early intervention with eculizumab, a complement C5 inhibitor, guided by pharmacokinetic/pharmacodynamic (PK/PD) model-informed precision dosing. However, not all patients respond to complement C5 inhibition in the same manner and may not be effectively treated using standard dosing recommendations. As such, targeted therapy is needed to more effectively treat TA-TMA patients. The instant disclosure addresses seeks to address one or more of the aforementioned needs in the art.

### BRIEF SUMMARY

Disclosed herein are methods of treating a human patient having TA-TMA, for example high-risk TMA with MODS or high-risk TMA without MODS, for example as characterized by elevated sC5b-9, comprising administration of a C5 inhibitor, for example eculizumab or an antigen binding fragment thereof. The disclosed dosing regimens may comprise one or more of a loading phase, induction phase, and maintenance phase, in particular, for patients who are “non-bleeding” patients, i.e., patients who do not have clinically relevant gastrointestinal bleeding.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 depicts the Optimal “Induction Dosing” Simulations of eculizumab for TA-TMA without gastrointestinal bleeding. Optimal dosing simulations were conducted by simulating eculizumab Ctrough using Monte Carlo Simulation approach based on our published population PK/PD model (Mizuno et al, Blood Adv 2022). The y-axis shows the simulated Ctrough of eculizumab, and the x-axis shows the body weight cohorts. The different colors represent different eculizumab mg dose amount from 300 mg to 1500 mg at 300 mg (1vial) increments. The simulated Ctrough in patients receiving FDA-approved q7d dosing regimen for aHUS showed a subtherapeutic eculizumab level (<100 μg/mL) for patients weighing ≥20 kg. (left panel). The q7d dosing regimen for patients weighing 20-<70 kg reached above the target Ctrough by increasing 300 mg (1 vial) (middle panel). Patients weighing ≥70 kg reached above the target by shortening the dosing interval to twice weekly (up to q4d) for the target attainment. (Right panel).

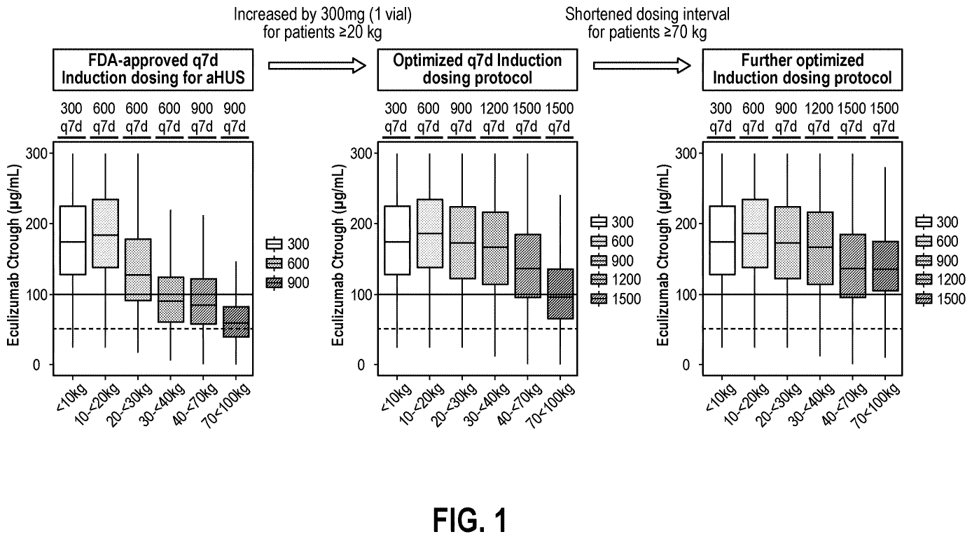

FIG. 2 depicts Optimal “Maintenance Dosing” Simulations of Eculizumab for TA-TMA. The y-axis shows simulated Ctrough of eculizumab based on our published model, and the x-axis shows the body weight cohorts. The different colors represent different eculizumab mg dose amount from 300 mg to 1200 mg at 300 mg (1 vial) increments. The horizonal dashed lines represent 50 and 75 μg/mL as potential target C_{trough}, levels for the maintenance therapy. Eculizumab PK/PD analysis demonstrated that the predicted eculizumab C_{trough} to suppress CH50 of 10% of baseline was 77 μg/mL (Mizuno et al, Clin Pharmacol Ther (2016), 99 (1), S18. PK/PD data was published by Jodele et al BBMT (2016), 22(2):307-315). Considering the goal of the maintenance therapy of sustained TA-TMA control and normal sC5b-9 levels for stopping the eculizumab therapy, optimal maintenance dosing protocols for more than 75% of patients to reach above 75 μg/mL were explored. The simulated Ctrough in patients receiving g14d dosing regimen with same mg dose amount of the protocol for aHUS showed subtherapeutic level for patients weighing ≥10 kg. (left panel). In patients weighing 10-<70 kg, the simulated Ctrough levels reached above 75 μg/mL for 75% of the simulated patients by shortening dosing interval from g14d to q7d (middle panel). In patients of the highest body weight group weighing 270 kg required 1200 mg twice weekly dosing for the target attainment. (right panel). Patients of the highest body weight group weighing 270 kg required twice weekly dosing for the target attainment. (Right panel).

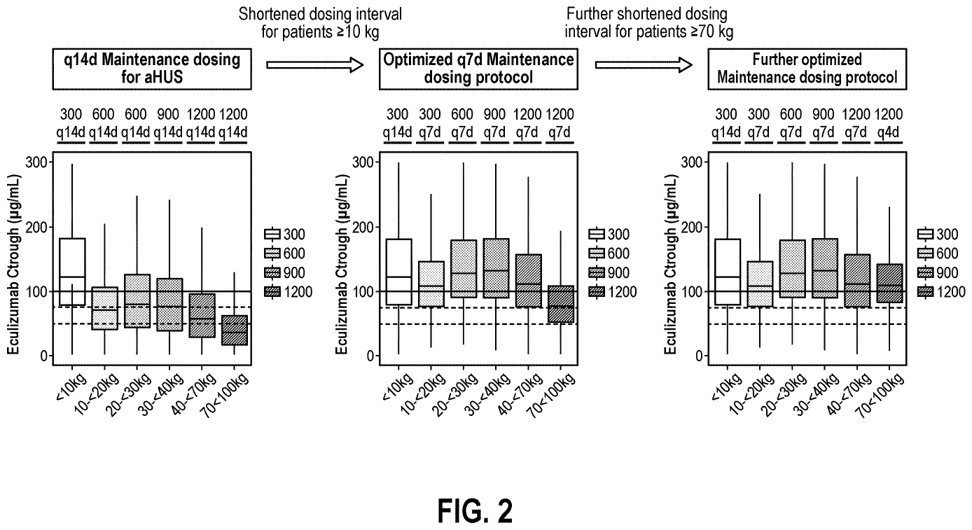

### DETAILED DESCRIPTION

### Definitions

Unless otherwise noted, terms are to be understood according to conventional usage by those of ordinary skill in the relevant art. In case of conflict, the present document, including definitions, will control. Exemplary methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “antibody fragment,” “antigen-binding fragment,” or similar terms refer to fragment of an antibody that retains the ability to bind to an antigen (e.g., a complement component C5 protein), e.g., a single chain antibody, a single chain Fv fragment (scFv), an Fd fragment, an Fab fragment, an Fab′ fragment, or an F(ab′)2 fragment. An scFv fragment is a single polypeptide chain that includes both the heavy and light chain variable regions of the antibody from which the scFv is derived. In addition, diabodies and intrabodies that bind to a complement component C5 protein can be incorporated into the compositions, and used in the methods, described herein.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

The methods may comprise, consist of, or consist essentially of the elements of the methods as described herein, as well as any additional or optional element described herein or otherwise useful in methods of treating an individual with transplant-associated thrombotic microangiopathy (TA-TMA, also referred to as HSCT-TMA) due to hematopoietic stem cell transplantation (HSCT).

The instant disclosure relates to complement C5 inhibitor, in particular eculizumab, pharmacokinetics/pharmacokinetics (PK/PD), and dosing regimens for complement inhibitor. Eculizumab is an C5 inhibitor sold under the tradename Soliris®, manufactured by Alexion Pharmaceuticals, Inc., Boston, MA, and is described in Kaplan (2002) Curr Opin Investig Drugs 3(7):1017-23; Hill (2005) Clin Adv Hematol Oncol 3 (11): 849-50; and Rother et al. (2007) Nature Biotechnology 25(11): 1256-1488.)

In one aspect, a method of treating a human patient with TA-TMA is disclosed. In one aspect, the method may comprise administering eculizumab or an antigen binding fragment thereof, to said individual according to Table 4, “Optimized eculizumab full dosing regimen for TA-TMA Patients (without clinically relevant bleeding).” In one aspect, the method may comprise treating a human patient with TA-TMA, comprising administering a loading dose of eculizumab or an antigen binding fragment thereof, to said individual according to Table 1 (“Dosing Option A (Q72 h loading dose regimen)”). In a further aspect, the method may comprise a method of treating a human patient with TA-TMA, comprising administering a loading dose of eculizumab or an antigen binding fragment to said individual according to Table 2 “Dosing Option B (More financially effective loading dose regimen with less mg per dose amount with flexible dosing intervals).”

In one aspect, the individual is one who does not have clinically significant bleeding. In one aspect, the TA-TMA may be high-risk TMA with MODS, or high-risk TMA without MODS, for example as characterized by elevated sC5b-9. The TA-TMA may be characterized by one or both of a sC5b-9 level that is at least twice the measured baseline level in said individual, or a sC5b-9 level that is greater than 244 nanograms per milliliter. In one aspect, the administration may be initiated at the time of TA-TMA diagnosis in said individual. The administration may be via intravenous administration.

In one aspect, the loading dose of the disclosed methods may be administered until normalization of sC5b-9 is achieved. In one aspect, the loading dose may be carried out until a normalized sC5b-9 is achieved, said normalized sC5b-9 being one or both of an sC5b-9 level of less than 244 ng/mL or an sC5b-9 level that is substantially at baseline (pre-transplant). In one aspect, the method may comprise an induction dose, said induction dose being administered until a hematological TA-TMA response selected from one or more of normalization of LDH, resolution of the need for red blood cell (RBC) and platelet transfusions, and disappearance of schistocytes is achieved. In one aspect, the administration may be carried out until a hematological TA-TMA response of normalization of LDH, resolution of the need for red blood cell (RBC) and platelet transfusions, and disappearance of schistocytes is achieved. In one aspect, the method may comprise a maintenance dose, wherein the maintenance dose may be administered until an eculizumab drug concentration (trough) of >100 μg/ml and/or an eculizumab drug concentration is rising with each dose while sC5b-9 remains normal.

### Loading Dose

The loading dose may be used to control complement activation by achieving normalization of sC5b-9. For example, an intensive loading dose may be used to normalize sC5b-9 as fast as possible by providing adequate eculizumab doses to fully suppress complement activity in blood as measured by total complement activity (CH50)<10% of normal laboratory value. The loading dose may use the maximum doses (mg) for patient weight, as TA-TMA clinical response is unlikely to be achieved unless sC5b-9 is normalized. In one aspect, the loading dose is carried out until sC5b-9 is normalized. In one aspect, eculizumab may be administered effective to normalize sC5b-9 within a two-week (14 day) period. In one aspect, eculizumab may be administered to a patient having elevated sC5b-9 for a period of about 11 to about 13 days. Elevated sC5b-9 may be one or both of >244 ng/ml or doubled as compared to the patient's baseline value.

In one aspect, the disclosed methods employ an interval dosing schedule. For example, in one aspect, each dose (mg) may be given at an interval ranging from about 24 hours to about 72 hours, for example, a 24 hour interval, or a 48 hour interval, or a 72 hour interval to a subject having elevated sC5b-9. In one aspect, the loading dose is administered for a period of time of at least about two weeks, or for a period of time of about two weeks. In one aspect, the loading dose may be a dose of from about 300 mg to about 1200 mg in an individual having a weight of <10 kg to >70 kg, or the dosing range may be about 300 mg given every 72 h for individual <10 kg, to about 1200 mg given every 24 hours for an individual 70 kg or more.

One exemplary loading dose regimen is set forth in Table 1. Dosing Option A (Q72h loading dose regiment). A further exemplary loading dose regimen is set forth in Table 2, “Dosing Option B (More financially effective loading dose regimen with less mg per dose amount with flexible dosing intervals)” which is particularly suited for minimizing cost associated with treatment, the administration optimized to efficiently use the commercially available eculizumab doses of 300 mg in one vial. Each of the foregoing loading dose schedules is particularly suited for the non-bleeding patient. A further exemplary loading dose regimen is set forth in Table 3, Dosing schedules for 24 weeks of Eculizumab Therapy. A further exemplary loading dose regimen is set forth in Table 4, Optimized eculizumab full dosing regimen for TA-TMA Patients (without clinically relevant bleeding). Each of the loading dose regimens may be used for an individual that is free of clinically relevant bleeding.

TABLE 1
Dosing Option A (Q72 h loading dose regimen)

| Weight | Loading dose (Q72 h × 2 weeks) |
|---|---|
| 70 kg or more | 2100 mg IV Q72 h × 5 (35 vials) |
| 40 kg to <70 kg | 1500 mg IV Q72 h × 5 (25 vials) |
| 30 kg to <40 kg | 1200 mg IV Q72 h × 5 (20 vials) |
| 20 kg to <30 kg | 900 mg IV Q72 h × 5 (15 vials) |
| 10 kg to <20 kg | 600 mg IV Q72 h × 5 |
| <10 kg | 300 mg IV Q72 h × 5 |

TABLE 2
Dosing Option B (More financially effective loading dose regimen
with less mg per dose amount with flexible dosing intervals).
It should be understood that the method may include a vial
size of about 300 mg, but may include vials of +/−10%
of the stated amount, or +/−5% of the stated amount.

| | Loading dose (flexible |
|---|---|
| Weight | dosing interval × 2 weeks) |
| 70 kg or more | 1200 mg IV Q24 h × 4 then |
| | Q72 h × 3 (28 vials) |
| 40 kg to <70 kg | 1200 mg IV Q48 h × 2 then |
| | Q72 h × 3 doses (20 vials) |
| 30 kg to <40 kg | 900 mg IV Q48 h × 2 then |
| | Q72 h × 3 doses (15 vials) |
| 20 kg to <30 kg | 600 mg IV Q48 h × 2 then |
| | Q72 h × 3 doses (10 vials) |
| 10 kg to <20 kg | 600 mg IV Q72 h × 5*¹ |
| <10 kg | 300 mg IV Q72 h × 5*² |

*¹When mg dose amount is reduced by 1 vial (300 mg) in patients <20 kg, the probability of target attainment falls significantly to ~40%.
*²The dose reduction was not considered in this body weight group due to the minimum dosage strength (300 mg/vial).

### Induction Dose

The disclosed induction dose schedule may be used to control sC5b-9 activation while achieving clinical TA-TMA response. In one aspect, the induction dosing time is for at least four weeks, or about four weeks. In one aspect, the total therapy time with loading and induction is at least about, or about six weeks, wherein the loading dose comprises about two weeks of the total loading dose and induction dose therapy period. In one aspect, the sC5b-9 activation is controlled with the loading dose period, such that when the induction dosing is started, the sC5b-9 level is normalized (less than about 244 ng/ml or substantially returned to patient baseline) when the induction dosing is started. In one aspect, the induction period is administered to a patient having a normalized sC5b-9 level and is administered every seven days. In one aspect, the patient has a weight of ≥70 kg, and is administered twice a week dosing during the induction period. In one aspect, the loading dose may be a dose of from about 300 mg/dose to about 1500 mg/dose in an individual having a weight of <10 kg to >70 kg, or in a further aspect, the dosing range may be about 300 mg given weekly for an individual <10 kg, to about 1500 mg given twice weekly for an individual 70 kg or more. Exemplary induction dose schedules are set forth in Tables 3 and 4, below.

As above, the induction dose schedules (including dosages and intervals) set forth herein are particularly suited for the non-bleeding patient.

### Maintenance Dose

Applicant found that eculizumab clearance in patients with TA-TMA was still higher by 23% in maintenance phase as compared to those with aHUS. Thus, the maintenance dosing period as disclosed herein may be used to effectively sustain TA-TMA control and normalized sC5b-9 levels, particularly in the non-bleeding patient. During the maintenance dosing period, complement activation may be assessed. In one aspect, when complement activation is resolved, eculizumab therapy can be safely stopped. In one aspect, the maintenance dosing phase is at least, or about 4 weeks. Maintenance dosing may be weekly for all weight groups except for those of >70 kg, in which the dose may be administered twice a week, and for those <10 kg, in which the dose may be given every other week.

In one aspect, the maintenance dose may be a dose of from about 300 mg/dose to 1200 mg/dose in an individual having a weight of <10 kg to >70 kg, or the dosing range may be 300 mg administered every other week for individual <10 kg to 1200 mg administered twice weekly for an individual 70 kg or more. In one aspect, the dosing schedule comprises a total dosing time of about 9.5 weeks, with about 5 to about 6 weeks of loading and induction therapy, and about 4 to about 5 of maintenance therapy. In one aspect, if, after four weeks of maintenance therapy, sC5b-9 remains normalized and eculizumab drug concentration (trough) is >100 μg/ml and/or is rising with each dose, then therapy can be discontinued. In one aspect, the maintenance phase is carried out until an eculizumab drug concentration (trough) of at least about 75 μg is achieved. In one aspect, the maintenance phase is carried out until an eculizumab drug concentration (trough) of at least about 80 μg is achieved. In one aspect, the maintenance phase is carried out until an eculizumab drug concentration (trough) of at least about 85 μg is achieved. In one aspect, the maintenance phase is carried out until an eculizumab drug concentration (trough) of at least about 90 μg is achieved. In one aspect, the maintenance phase is carried out until an eculizumab drug concentration (trough) of at least about 100 μg is achieved.

Exemplary maintenance dose schedules are set forth in Table 3 and 4. Each may be particularly useful for treatment of a non-bleeding patient.

In one aspect, the dosing regimen may comprise a loading dose phase, comprising at least one loading dose, an induction dose phase, comprising at least one induction dose, and a maintenance dose phase, comprising at least one maintenance dose. For example, in one aspect, the method may comprise administration of eculizumab according to the following Table 3, having each of the aforementioned phases:

TABLE 3
Dosing schedules for 24 weeks of Eculizumab Therapy

| Weight | Loading Dose Phase | Induction Dose Phase | Maintenance Dose Phase |
|---|---|---|---|
| 40 kg or more | 900 mg IV Q48 h × 2 | 900 mg IV Q7 d × 4 | 1200 mg IV Q14 d × 9 |
| | then Q72 h × 3 doses | | |
| 30 kg to <40 kg | 600 mg IV Q48 h × 2 | 600 mg IV Q7 d × 4 | 900 mg IV Q14 d × 9 |
| | then Q72 h × 3 doses | | |
| 20 kg to <30 kg | 600 mg IV Q48 h × 2 | 600 mg IV Q7 d × 4 | 600 mg IV Q14 d × 9 |
| | then Q72 h × 3 doses | | |
| 10 kg to <20 kg | 600 mg IV Q48 h × 2 | 600 mg IV Q7 d × 4 | 300 mg IV Q14 d × 9 |
| | then Q72 h × 3 doses | | |
| <10 kg | 300 mg IV Q48 h × 2 | 300 mg IV Q7 d × 4 | 300 mg IV Q14 d × 9 |
| | then Q72 h × 3 doses | | |

In a further aspect, the method may comprise administration of eculizumab according to the following Table 4, having each of a loading dose phase, an induction dose phase, and a maintenance dose phase.

TABLE 4
Optimized eculizumab full dosing regimen for TA-
TMA Patients (without clinically relevant bleeding)

| | Loading dose phase | Induction dose phase | Maintenance phase |
|---|---|---|---|
| Weight | (2 weeks) | (4 weeks) | (4 weeks) |
| 70 kg or more | 1200 mg IV Q24 h × 4 | 1500 mg IV twice | 1200 mg IV twice |
| | then Q72 h × 3 doses | weekly | weekly |
| 40 kg to <70 kg | 1200 mg IV Q48 h × 2 | 1500 mg IV weekly | 1200 mg IV weekly |
| | then Q72 h × 3 doses | | |
| 30 kg to <40 kg | 900 mg IV Q48 h × 2 | 1200 mg IV weekly | 900 mg IV weekly |
| | then Q72 h × 3 doses | | |
| 20 kg to <30 kg | 600 mg IV Q48 h × 2 | 900 mg IV weekly | 600 mg IV weekly |
| | then Q72 h × 3 doses | | |
| 10 kg to <20 kg | 600 mg IV Q72 h × 5 | 600 mg IV weekly | 300 mg IV weekly |
| | doses | | |
| <10 kg | 300 mg IV Q72 h × 5 | 300 mg IV weekly | 300 mg IV every 2 |
| | doses | | weeks |

In one aspect, the administering may be carried out over a 24-week administration cycle. For example, in one aspect, the administration cycle may comprise:
a loading dose of 300 milligrams to a patient weighing <10 kilograms, or 600 milligrams to a patient weighing 10 kilograms to less than 40 kilograms, or 900 milligrams to a patient weighing 40 kilograms or more, said loading dose being administered or more every 48 hours times 2, then every 72 hours times 3;
an induction dose, wherein said induction dose follows said loading dose, of 300 milligrams to a patient weighing <10 kilograms, or 600 milligrams to a patient weighing 10 kilograms to less than 40 kilograms, or 900 milligrams to a patient weighing 40 kilograms or more, said induction dose being administered every seven days times 4; and
a maintenance dose, wherein said maintenance dose follows said induction dose, of 300 milligrams to a patient weighing <10 kilograms to <20 kilograms, or 600 milligrams to a patient weighing 20 kilograms to less than 30 kilograms, or 900 milligrams to a patient weighing 30 kilograms to less than 40 kilograms, or 1200 milligrams to a patient weighing 40 kilograms or more, said maintenance dose being administered every 14 days times 9.

In one aspect, the administration cycle may comprise:
a loading dose of eculizumab comprising
   about 300 milligrams every 72 hours for five doses when said individual weighs less than 10 kilograms;
   about 600 milligrams every 72 hours for five doses when said individual weighs 10 kilograms to less than 20 kilograms;
   about 600 milligrams every 48 hours for two doses, and about 600 milligrams intravenously every 72 hours for three doses, when said individual weighs 20 kilograms to less than 30 kilograms;
   about 900 milligrams every 48 hours for two doses, and about 900 milligrams intravenously every 72 hours for three doses, when said individual weighs 30 kilograms to less than 40 kilograms;
   about 1200 milligrams every 48 hours for two doses, and about 1200 milligrams every 72 hours for three doses, when said individual weighs 40 kilograms to less than 70 kilograms; or
   about 1200 milligrams every 24 hours for four doses, and about 1200 milligrams every 72 hours for three doses, when said individual weighs 70 kilograms or more;
an induction dose of eculizumab comprising
   about 300 milligrams weekly when said individual weighs less than 10 kilograms;
   about 600 milligrams weekly when said individual weighs 10 kilograms to less than 20 kilograms;
   about 900 milligrams weekly when said individual weighs 20 kilograms to less than 30 kilograms;
   about 1200 milligrams weekly when said individual weighs 30 kilograms to less than 40 kilograms;
   about 1500 milligrams weekly when said individual weighs 40 kilograms to less than 70 kilograms; or
   about 1500 milligrams twice weekly, when said individual weighs 70 kilograms or more; and
a maintenance dose of eculizumab comprising
   about 300 milligrams every two weeks when said individual weighs less than 10 kilograms;
   about 300 milligrams weekly when said individual weighs 10 kilograms to less than 20 kilograms;
   about 600 milligrams weekly when said individual weighs 20 kilograms to less than 30 kilograms;
   about 900 milligrams weekly when said individual weighs 30 kilograms to less than 40 kilograms;
   about 1200 milligrams weekly when said individual weighs 40 kilograms to less than 70 kilograms; or
   about 1500 milligrams twice weekly, when said individual weighs 70 kilograms or more;
said loading dose being carried out for a two-week period;
said induction dose being carried out for a four-week period;
said maintenance dose being carried out for a four-week period; and
said administration being intravenous administration.

In one aspect, the administration cycle may comprise:
a loading dose of eculizumab comprising:
   a dose of about 300 milligrams every 72 hours for an individual weighing less than 10 kilograms;
   a dose of about 300 milligrams every 72 hours for five doses for an individual weighing 10 kilograms to <20 kilograms;
   a dose of about 600 milligrams every 72 hours for five doses for an individual weighing 10 kilograms to <20 kilograms;
   a dose of about 900 milligrams every 72 hours for five doses for an individual weighing 20 kilograms to <30 kilograms;
   a dose of about 1200 milligrams every 72 hours for five doses for an individual weighing 30 kilograms to <40 kilograms;
   a dose of about 1500 milligrams every 72 hours for five doses for an individual weighing 40 kilograms to <70 kilograms; or
   a dose of about 2100 milligrams every 72 hours for five doses for an individual weighing 70 kilograms or more;
said administration being intravenous administration.

In some aspects, the patient has not previously been treated with a complement inhibitor (e.g., the patient is a complement inhibitor treatment-naïve patient). In one aspect, the patient may have been previously treated with eculizumab but have no detectable or below therapeutic drug level and may be re-treated again.

In some aspects, the individual is an infant. The individual may be, e.g., 0.5 (e.g., 1, 1.5, 2, 2.5, 3, 3.5, 4, 4.5, 5, 5.5, 6, 6.5, 7, 7.5, 8, 8.5, 9, or 9.5) years old. The infant can be less than 10 (e.g., less than 9.5, 9, 8.5, 8, 7.5, 7, 6.5, 6, 5.5, 5, 4.5, 4, 3.5, 3, 2.5, 2, 1.5, 1, or less than 1) year(s) old. In one aspect, the individual is a neonate (from birth through the first 28 days of life), or an infant (29 days of age to less than 2 years of age), a child (2 years of age to less than 12 years of age) or an adolescent (aged 12 through 21, up to but not including the 22nd birthday). In some aspects, the individual may be an adult.

### EXAMPLES

The following non-limiting examples are provided to further illustrate embodiments of the invention disclosed herein. It should be appreciated by those of skill in the art that the techniques disclosed in the examples that follow represent merely exemplary aspects of the disclosure.

### Example 1

### Methods

### Study Subjects

Study parameters and methods are described in Eculizumab precision-dosing algorithm for thrombotic microangiopathy in children and young adults undergoing HSCT. Mizuno K, Dandoy C E, Teusink-Cross A, Davies S M, Vinks A A, Jodele S. Blood Adv. 2022 Mar. 8; 6(5):1454-1463. doi: 10.1182/bloodadvances.2021006523.PMID: 35008105. In brief, a clinical cohort of sixty-four patients with high-risk TA-TMA treated with eculizumab was available for analysis. All study subjects were prospectively and uniformly monitored for TA-TMA and underwent real-time eculizumab PK/PD monitoring. Clinical outcomes of this cohort were published in Blood by Jodele et al, Complement blockade for TA-TMA: lessons learned from a large pediatric cohort treated with eculizumab. Jodele S, Dandoy C E, Lane A, Laskin B L, Teusink-Cross A, Myers K C, Wallace G, Nelson A, Bleesing J, Chima R S, Hirsch R, Ryan T D, Benoit S, Mizuno K, Warren M, Davies SM.Blood. 2020 Mar. 26; 135(13):1049-1057. doi: 10.1182/blood.2019004218.PMID: 31932840. Patients with any clinical evidence of lower intestinal bleeding were marked as having clinically significant bleeding and were assigned to the bleeding patient group for analysis.

The details of eculizumab dosing, response monitoring methods, and clinical outcomes assessment are described in Jodele S, Dandoy C E, Lane A, et al. Complement blockade for TA-TMA: lessons learned from a large pediatric cohort treated with eculizumab. Blood. 2020; 135(13):1049-1057.

Eculizumab dosing methods are currently not available for HSCT recipients with TA-TMA. Clinicians have assumed that TA-TMA is a similar disease state to that of aHUS and have applied aHUS-approved eculizumab dosing methods for aHUS. This dosing strategy has led to inadequate clinical responses in TA-TMA patients, and assumptions that eculizumab is not effective in TA-TMA. Applicant has demonstrated that TA-TMA is a specific disorder within the group of thrombotic microangiopathies (TMAs) that has unique clinical presentations of organ injury, and specific, previously not reported, responses to complement blocking therapy, namely eculizumab. Applicant has identified eculizumab drug clearance patterns specific for patients with high-risk complement mediated TA-TMA, for whom the disclosed, optimized eculizumab dosing regimens may be used. These optimized dosing regimens result in significant improvement in clinical outcomes in this lethal condition, particularly as compared to the aHUS dosing strategy that had previously been used. While precision dosing at point of care based on real-time PK assays are ideal, PK/PD model-informed “fixed” eculizumab dosing regimens developed for different body weight groups are needed for treating patients for whom real time drug PK assays are not available. Such dosing schedules are particularly useful for non-bleeding patients, as bleeding patients require different regimens.

The disclosed eculizumab dosing strategies are novel, not previously reported by others and not intuitive based on clinical approaches in other TMAs, including aHUS. The TA-TMA therapy and outcomes data was derived from 130 patients with TA-TMA (64 eculizumab treated high risk patients, 48 moderate risk untreated TA-TMA and 18 low risk TA-TMA patients). Clinical conclusions on eculizumab PK cannot be sufficiently or intuitively drawn from random patient cases or small patient cohorts as, based on Applicant's unique observations, eculizumab drug clearance has a wide variability within and between patients, depending on TA-TMA disease activity and body weight.

Eculizumab is dosed based on patient weight (kg) and is provided by the manufacturer as 300 mg vials for intravenous use. Applicant showed that HSCT recipients with TA-TMA receiving eculizumab can benefit from more precise weight range grouping than proposed for aHUS (see Table 5) for eculizumab dosing to avoid underdosing (larger patients) or overdosing (smaller patients). Applicant also demonstrated that patient body weight and sC5b-9 levels are significant determinants of eculizumab kinetics, and the eculizumab dosing is optimized depending on patients' body weight and sC5b-9 levels. With the assumption of similar sC5b-9 distribution as observed in our TA-TMA patient cohort, the eculizumab dosing may require higher dosing for TA-TMA patients 40 kg or larger for weekly or twice weekly dosing. Such increased dosing is contrary to the current eculizumab drug label for aHUS, in which the maximum single dose of eculizumab given is 1200 mg. Applicant also previously demonstrated that high eculizumab levels in blood is safe and provides adequate complement blockade in TA-TMA. Optimized eculizumab, full dosing regimen for individuals without clinically relevant bleeding, is shown above, in Table 4.

TABLE 5
FDA-approved eculizumab dosing regimen labeled for aHUS

| Weight | Induction dose | Maintenance dose |
|---|---|---|
| 40 kg or more | 900 mg IV | 1200 mg at week 5; then |
| | weekly × 4 doses | 1200 mg every 2 weeks |
| 30 kg to <40 kg | 600 mg IV | 900 mg at week 3; then |
| | weekly × 2 doses | 900 mg every 2 weeks |
| 20 kg to <30 kg | 600 mg IV | 600 mg at week 3; then |
| | weekly × 2 doses | 600 mg every 2 weeks |
| 10 kg to <20 kg | 600 mg IV | 300 mg at week 2; then |
| | weekly × 1 dose | 300 mg every 2 weeks |
| 5 to <10 kg | 300 mg IV | 300 mg at week 2; then |
| | weekly × 1 dose | 300 mg every 3 weeks |

### Loading Dose

Applicant demonstrated that elevated sC5b-9 is a high-risk TA-TMA marker and is associated with a three-fold higher risk of mortality and overall worse outcomes. Subjects with elevated sC5b-9 above 244 ng/mL or doubled from the baseline are at increased risk for multi-organ injury syndrome (MODS). Sustained sC5b-9 activation for >2 weeks results in multi-organ injury in patients with TA-TMA. Transplantation-Associated Thrombotic Microangiopathy Risk Stratification: Is There a Window of Opportunity to Improve Outcomes?, Jodele S, Dandoy C E, Sabulski A, Koo J, Lane A, Myers K C, Wallace G, Chima R S, Teusink-Cross A, Hirsch R, Ryan T D, Benoit S, Davies; Variable Eculizumab Clearance Requires Pharmacodynamic Monitoring to Optimize Therapy for Thrombotic Microangiopathy after Hematopoietic Stem Cell Transplantation; Jodele S, Fukuda T, Mizuno K, Vinks A A, Laskin B L, Goebel J, Dixon B P, Chima R S, Hirsch R, Teusink A, Lazear D, Lane A, Myers K C, Dandoy C E, Davies S M. Biol Blood Marrow Transplant. 2016 February; 22(2):307-315. doi: 10.1016/j.bbmt.2015.10.002. Epub 2015 Oct. 9.PMID: 26456258.

By using the disclosed eculizumab personalized dosing strategies, Applicant demonstrated that normalization of significantly elevated sC5b-9 may be achieved in less than 2 weeks. This was achieved, in part, by administering an intensive eculizumab loading regimen during first two weeks of therapy to control complement activation as fast as possible to avoid organ injury and death, a concept not evident from nor proposed in patients receiving eculizumab for aHUS.

Applicant also identified that weight-based eculizumab dose is ideally given in intervals no longer than 72 hours in subjects with elevated sC5b-9, or in some instances, in intervals no longer than every 48 hours for patients of 20 kg to <70 kg and in intervals no longer than every 24 hours for patients of 70 kg or more to successfully block complement activity and to achieve desired clinical TA-TMA response. Applicant simulated the probability of target attainment by dosing interval reduction depending on patient's weight and drug clearance to determine the precise dose (in mg) and the dosing interval needed for optimal complement blockade and to determine the shortest time to normalization of elevated sC5b-9. Based on the simulations, patients with the largest weight (>70 kg) required high dose (mg) for each infusion even with the 24 hour dosing interval. As such, when the dosing interval could not be reasonably shortened, the dose (mg) was increased at 300 mg increments (1 vial) to achieve and to sustain therapeutic drug concentration and clinical effect. Eculizumab precision-dosing algorithm for thrombotic microangiopathy in children and young adults undergoing HSCT. Mizuno K, Dandoy C E, Teusink-Cross A, Davies S M, Vinks A A, Jodele S. Blood Adv. 2022 Mar. 8; 6(5):1454-1463. doi: 10.1182/bloodadvances.2021006523.PMID: 35008105.

Different optimal dosing regimens were derived either using fixed administration interval (in hours) or fixed weight-based dose (in mg) or as summarized by Mizuno et al Blood Adv. 2022 Mar. 8; 6(5):1454-1463 to determine the most clinically and financially optimal dosing regimen for the loading phase, and later for the induction and maintenance phases.

The tables displayed below show the comparison of OPTION A and OPTION B using multiple factors such as disease response, drug pharmacokinetics and amount of drug needed. The dosing tables are inclusive of the financial aspect of eculizumab therapy, wherein a dosing schedule may be selected to minimize financial impact while optimizing the therapeutic outcome.

“Dosing option A” was determined using the fixed dosing interval of 72 h by escalating dose given (mg) to achieve and sustain therapeutic eculizumab drug concentration trough of ≥100 μg/ml. “Dosing option B” was determined using fixed dose (mg) for weight groups and adjusting the dosing interval to achieve and sustain a therapeutic eculizumab drug concentration trough of ≥100 μg/ml. Options A and B were compared by how much drug was required as it is dosed in 300 mg vial increments for a full loading dosing course to determine a more financially effective regimen for the same drug concentration and clinical response achieved. Option B required fewer drug vials (and therefore is more economically feasible) to achieve the same response and may therefore be chosen as priority regimen. Option A may be suggested as an alternative if the “fixed” dosing interval is preferred (for example, to accommodate patient availability and to allow for medical visits every three days instead of daily).

The tables showing “Loading Dose” Options for Eculizumab for treatment of TA-TMA as developed by Applicant are shown above as Tables 1 and 2.

### Induction Dose

Applicant determined that the induction dosing phase should be continued until clinically significant control/resolution of TA-TMA is achieved and sC5b-9 remains normal under complement blockade control. Applicant identified an eculizumab dosing regimen for the induction phase by using eculizumab PK data from clinically treated patients with high-risk TA-TMA (n=64) in which superior survival was demonstrated (66% vs 16.7% 1 year post transplant survival) as compared to same risk patients not receiving complement blocking therapy. See Complement blockade for TA-TMA: lessons learned from a large pediatric cohort treated with eculizumab. Jodele S, Dandoy C E, Lane A, Laskin B L, Teusink-Cross A, Myers K C, Wallace G, Nelson A, Bleesing J, Chima R S, Hirsch R, Ryan T D, Benoit S, Mizuno K, Warren M, Davies S M. Blood. 2020 Mar. 26; 135(13):1049-1057. doi: 10.1182/blood.2019004218.PMID: 31932840. Applicant found that TA-TMA patients exhibited higher drug clearance as compared to those reported with aHUS patients for at least 5 weeks after clinical disease presentation. Applicant found that a loading and induction therapy having a total period of 6 weeks as obtaining the best clinical response and identified the optimal drug dose (mg) for weekly drug administration for the induction phase of therapy for patients <70 kg. Patients ≥70 kg had better drug clearance curves with twice a week dose administration; accordingly, this is the only weight group proposed to benefit from twice a week dosing in the induction dosing phase.

### Maintenance Dosing

The maintenance dosing phase allows for the shortest therapy time required before eculizumab therapy can be ceased after the TA-TMA process is controlled with the loading and induction dosing phases, with sC5b-9 remaining controlled. Using high intensity and brief therapy for TA-TMA is a novel concept because the recommended eculizumab therapy for aHUS is indefinite administration. Applicant found that 4-5 weeks of maintenance therapy was required for patients with TA-TMA to safely discontinue the eculizumab treatment. Disclosed is a novel strategy of using the eculizumab drug level to determine resolution of complement activation and maintenance phase length. Applicant proposed that if during 4 weeks of maintenance therapy sC5b-9 remains normal and eculizumab drug concentration (trough) of >100 μg/ml and/or if eculizumab drug concentration is rising with each dose, then the therapy may be safely discontinued. In other aspects, maintenance dosing that maintains about 75 μg/ml is sufficient to sustain complement inhibition, and the maintenance dosing may be administered in at a dosing interval or amount sufficient to achieve such level. The optimized maintenance dosing regimen for each weigh group provides decided clinical benefit with the least amount of drug (mg) used. Previously unreported findings showed that eculizumab clearance in patients with TA-TMA was still higher by 23% in maintenance phase as compared to those with aHUS. Applicant thus proposed a weekly drug dosing option for all weight groups except for those >70 kg that required twice weekly dosing and those <10 kg who only required dosing every 2 weeks using a weight-based drug (mg) dose to sustain a therapeutic eculizumab drug level of ≥75 μg/ml resulting in desired clinical response. This strategy provides precision eculizumab dosing for clinical care by determining the exact patient needs for complement blocking therapy and provides not only clinical benefit but also the most efficient use of the drug, minimizing financial impact.

The resulting eculizumab dosing regimen including loading (2 weeks), induction (4 weeks), and maintenance (4 weeks) dosing for TA-TMA derived from personalized drug clearance pharmacokinetics provides a novel strategy for patients with TA-TMA, and provides targeted drug administration to achieve an improved clinical response and best financial option, thereby minimizing the expense and length of treatment, particularly in the clinical settings where real-time drug PK data is not available.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. All accessioned information (e.g., as identified by PUBMED, PUBCHEM, NCBI, UNIPROT, or EBI accession numbers) and publications in their entireties are incorporated into this disclosure by reference in order to more fully describe the state of the art as known to those skilled therein as of the date of this disclosure. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

1. A method of treating an individual with Transplant-Associated Thrombotic Microangiopathy (TA-TMA), comprising administering eculizumab or an antigen binding fragment thereof, to the individual according to Table 4 (Optimized Eculizumab Full Dosing Regimen for TA-TMA).

2. A method of treating an individual with TA-TMA, comprising administering a loading dose of eculizumab or an antigen binding fragment thereof, to the individual according to Table 1 (Dosing Option A).

3. A method of treating an individual with TA-TMA, comprising administering a loading dose of eculizumab or an antigen binding fragment to the individual according to Table 2 (Dosing Option B).

4. The method of claim 1, wherein a loading dose is administered until normalization of sC5b-9 is achieved.

5. The method of claim 1, where the TA-TMA is selected from high-risk TMA with Multiorgan Injury Syndrome (MODS) or high-risk TMA without MODS.

6. The method of claim 1, wherein the TA-TMA is characterized by a sC5b-9 level that is at least twice a measured baseline level in the individual.

7. The method of claim 1, wherein the TA-TMA is characterized by a sC5b-9 level that is greater than 244 nanograms per milliliter.

8. The method of claim 1, wherein the administering is initiated at TA-TMA diagnosis in the individual.

9. The method of claim 1, wherein the administering is intravenous administration.

10. The method of claim 1, wherein a loading dose is carried out until a normalized sC5b-9 is achieved, said normalized sC5b-9 being one or both of an sC5b-9 level of less than 244 ng/mL or an sC5b-9 level that is substantially at baseline (pre-transplant).

11. The method of claim 1, wherein the method comprises an induction dose, the induction dose being administered until a hematological TA-TMA response selected from one or more of normalization of LDH, resolution of a need for red blood cell (RBC) and platelet transfusions, and disappearance of schistocytes is achieved.

12. The method of claim 1, wherein the administering is carried out until a hematological TA-TMA response of normalization of LDH, resolution of a need for red blood cell (RBC) and platelet transfusions, and disappearance of schistocytes is achieved, the administering comprising an induction dose and a loading dose.

13. The method of claim 1, comprising a maintenance dose, wherein the maintenance dose is administered until an eculizumab drug concentration (trough) of at least about 75 μg/ml is achieved.

14. The method of claim 1, comprising a maintenance dose, wherein the maintenance dose is administered until an eculizumab drug concentration (trough) of at least about 100 μg/ml is achieved.

15. The method of claim 1, comprising a maintenance dose, wherein the maintenance dose is administered until an eculizumab drug concentration is rising with each dose while sC5b-9 remains normal.

16. The method of claim 1, wherein the individual does not have clinically significant bleeding.

17. The method of claim 1, wherein the individual is an infant.

18. The method of claim 1, wherein the individual is pre-pubescent.

19. The method of claim 1, wherein the individual is an adult.
